(19) United States
(12) Patent Application Publication
(10) Pub. No.: US 2017/0319047 A1
(43) Pub. Date: Nov. 9, 2017

(54) DEVICE FOR USE IN HYSTEROSCOPY

(71) Applicant: Lina Medical APS, Glostrup (DK)

(72) Inventors: Henrik Bisgaard POULSEN, Slangerup (DK); Anders RASMUSSEN, Copenhagen V (DK); Jonas ANDERSEN, Frederiksberg (DK)

(21) Appl. No.: 15/527,665

(22) PCT Filed: Nov. 17, 2015
(86) PCT No.: PCT/EP2015/076863

§ 371 Date: May 17, 2017

(30) Foreign Application Priority Data
Nov. 17, 2014 (DK) PA 2014 70707

(51) Int. Cl. A61B 1/00; A61B 1/005; A61B 1/05; A61B 1/303

(52) U.S. Cl. CPC A61B 1/0008; A61B 1/303; A61B 1/005; A61B 1/05

(57) ABSTRACT

A hysteroscopy device with an image capturing structure located at a distal end of an elongated member and communicating video signals with a monitor. The elongated member and the image capturing structure being dimensioned for insertion into the patient's uterus through cervix.

### INTRODUCTION

The present invention relates to a portable device for use in hysteroscopy.

### BACKGROUND

Gynecologists use hysteroscopy in standard examination procedures, both in office-based procedures and in hospital procedures. In hysteroscopy, an endoscope is inserted into the uterus e.g. for inspecting the lining.

For the practitioner, the field of diagnostic imaging, for example hysteroscopy, has allowed viewing of the internal lining in uterus with minimal complication and pain.

Such imaging tools have been used in different forms for detailed different kinds of inspection.

In the medical field, the large amount of permanently or semi-permanently installed devices is challenging. The costs for purchasing and costs for maintenance of the equipment, and the complexity necessitate skills and training for the staff.

Additionally, the technical equipment takes up space, they require fixed power supply, and/or supply of fluids etc., and they may be impossible to move close to the practitioner during surgery and therefore they may sometimes be in the way for the staff and they can be difficult to use.

Sterility and re-useability are closely related. The fixtures of an operating room must typically be clean or even sterile before they can be used. Sterility can be accomplished by using a device only once, but typically, the large electrical fixtures including endoscopes and monitors of an operating room are much too expensive to be used only once.

The existing endoscopes are typically two-unit devices made of a relatively inexpensive scope with a camera or with fiber optics, and a very expensive control unit forming part of the fixture of an operating room. The scope and control unit are connected by use of cables, e.g. including optical cables. The existing endoscopes are complicated to use and require adaption of settings between the scope and the control unit. Additionally, sterility can be compromised when the non-sterile control unit is connected to the sterile scope by cables.

Integrated endoscopic devices exist where the camera is directly connected to a control unit with a monitor. Such devices are simple to use but typically provide a limited set of operating features as compared with the known two unit devices.

### DESCRIPTION OF THE INVENTION

It is an object of the invention to improve sterility, safety of operation, and simplicity of devices for hysteroscopy and to reduce the risk of having wrong settings, bad connectivity, or lack of compatibility between releasably connected units of a device. It is a further object to ease insertion and to improve hysteroscopy in combination with use of tools inserted through the hysteroscope.

The invention, in a first aspect, provides a device for visualization of internal tissue of a patient's uterus, the device comprising a hand-held control unit, an elongated member, and an image capturing structure.

The control unit is dimensioned to be held by a user's hand and it may include various components such as a monitor.

The elongated member has a proximal end connected to or connectable to the hand-held control unit and a distal end holding the image capturing structure.

The image capturing structure is configured to communicate video signals with a monitor, and at least the distal end of the elongated member and the image capturing structure is dimensioned for insertion into the patient's uterus through cervix.

Since the control unit is dimensioned to be held by a user's hand and since the elongated member is connected to or connectable to the control unit, the control unit may be used without connection to any external devices. The device according to the invention therefore becomes easy to use directly upon removal from the package without having to assembly cables or attached external camera or monitor. This reduces the risk of errors, reduces the risk of combining non-compatible items, and reduces the risk of contaminating the device during connection to external components.

The control unit may particularly be independently powered by a battery, and it may be completely fitted with any necessary parts such as a monitor, fluid flushing systems and other parts which are suitable for the procedure. In that way, the device may form a complete, independent, hysteroscopy device, e.g. suitable for single usage.

The elongated member extends along a longitudinal axis and may particularly form an internal elongated conduit forming space for cables, tubes and tools inside the elongated member. Particularly, the internal conduit may extend along the longitudinal axis from a proximal opening, e.g. formed in, or by, the control unit, to a distal opening at or near the distal end of the elongated member. The device may further comprise such a tool which is movable within the elongated member.

The distal portion of the elongated member may include an S-shaped portion configured to hold the image capturing structure in an offset position relative to a longitudinal axis of the elongated member. Due to the S-shaped portion, the image capturing structure is in a position that allows an easier passage of a tool through the internal elongated conduit, and it may allow the tool and the image capturing structure to be placed side by side, e.g. in a parallel configuration, during surgery.

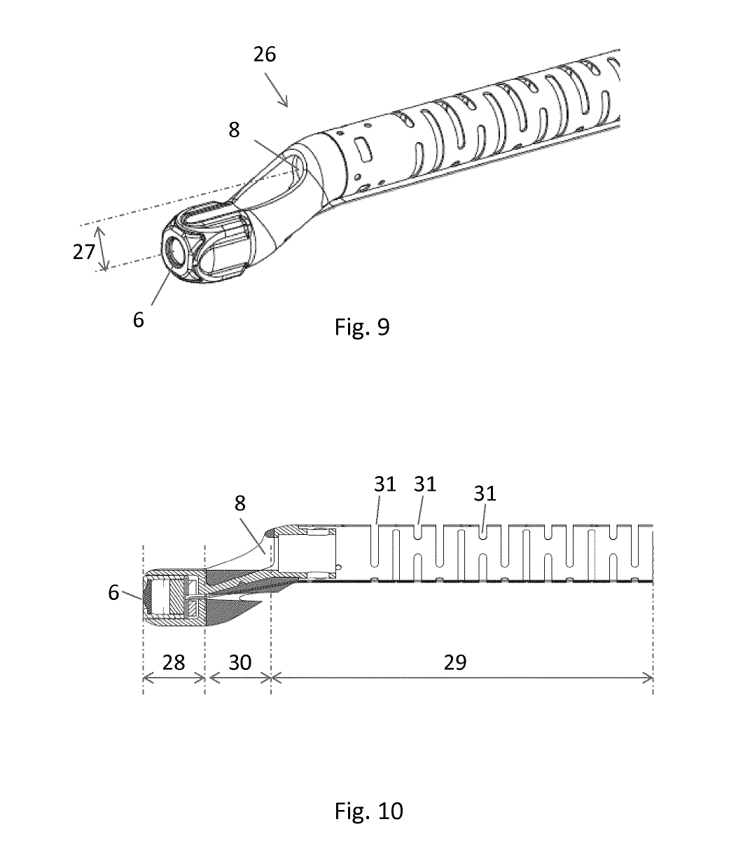

By S-shape is herein meant any shape which can be split into two outer sections (a forward and a rearward S-portion) and an intermediate section (intermediate S-portion), where the intermediate section is transverse to the outer sections. Examples of shapes which are within the definition of S-shape are illustrated in FIGS. 9-13 and described relative to specific examples.

The image capturing structure may be held in a position where it partially hinders passage of the tool or in another configuration where the image capturing structure does not hinder the passing of the tool through the internal conduit. Particularly, the distal opening may be in the S-shaped portion.

The elongated member may comprise a proximal portion forming the proximal end and a distal portion forming the distal end, the proximal portion and the distal portion having different bending characteristics. I.e. the elongated member may e.g. have different stiffness along the length of the elongated member. Particularly it may form a rigid proximal portion near the control unit and a more resilient, flexible, or bendable portion towards the distal end—i.e. further away from the control unit.

The elongated member could be soft and adaptable to the shape of the cervix and uterus, however, it is also an object to maintain a firm connection between the image capturing structure and the control unit so that movement with the handle corresponds to movement of the image capturing structure. Therefore a shaft with different bending characteristics will provide control over the image capturing structure for the practitioner and a safer and more comfortable procedure for the patient.

The S-shaped portion may be formed at least partly by the distal portion.

The S-shaped portion may be formed by a forward S-portion and a rearward S-portion, the forward S-portion and the rearward S-portion having different bending characteristics. The forward and rearward S-portions could e.g. be separated by the aforementioned intermediate section being between the outer sections in the definition of S-shape.

The distal opening could be between the forward S-portion and the rearward S-portion.

The proximal portion may be linear, e.g. to form a straight line from the control unit all the way to the S-shaped portion.

The distal portion may be made from a material selected from the group consisting of: SEBS, PUR, and EVA.

The distal portion may have a length being less than 30 mm.

In one embodiment the distal section is more elastically deformable than the proximal section. This will make the parts that are introduced into the uterus more elastically deformable in order to be more adaptable to the shape of the uterus cavity and thus reduce discomfort during the procedure and reduce the risk of damaging tissue.

In one embodiment the forward S-portion is more elastically deformable than the rearward S-portion. This may make the distal end more adaptable to the cavity and will specifically reduce discomfort during introduction of the elongated member through cervix and reduce the risk puncturing the uterus.

Further parts of the elongated member may comprise two different materials, e.g. having a core which is more rigid than an outer layer.

The elongated member may have a generally uniform cross-section throughout its length.

In order to reduce discomfort for the patient during introduction of the instrument through cervix it is an advantage that the instrument is as small as possible, further it is desired that the surface and geometry is generally smooth in order to make the instrument adapt as good as possible during introduction. To reduce discomfort during the introduction of the elongated member e.g. through cervix, the outer diameter of the elongated member may be reduced towards the distal end.

The elongated conduit may particularly be made such that it can hold a tool in a fixed position relative to the image capturing structure, e.g. parallel to an axis defined by a portion of the elongated member, particularly the proximal portion.

The distal end of the elongated member may define a plane which is perpendicular to the center axis of the proximal portion of the elongated member, and the S-shaped portion may form a curved course between said plane and the proximal portion.

The image capturing structure may be movable relative to the distal end of the elongated member. This will allow better adaptation of the device to the specific size and/or shape of the uterus and/or cervix of the patient and the capturing structure may e.g. be displaced during use to provide better space for other surgical instruments, e.g. for instruments dedicated for taking tissue samples, for electrosurgical instruments e.g. for cutting or coagulating tissue, for thermal ablation instruments, or for surgical or electrosurgical forceps.

Particularly, the image capturing structure may be movable between a first position in which it at least partly hinders passage through the elongated conduit and a second position providing unhindered passage through the elongated conduit.

The image capturing structure may e.g. be located inside an axial bore in the distal end of the elongated member, and the bore could be made to obtain the claimed movability of the image capturing structure relative to the distal end, e.g. by providing the bore with a larger cross section than the cross section of the image capturing means such that the image capturing means can move radially relative to a longitudinal axis of the elongated member.

The axial bore may be separate from the internal conduit or it may form a distal end portion of the internal conduit. It may e.g. be constituted by a portion of the internal conduit where the conduit is widened out and defines an increased cross sectional area.

The axial bore may foul′ a radial opening allowing the image capturing structure to move out of the bore in a radial direction relative to the longitudinal axis.

The radial opening could be in the form of a slot or recess. In one embodiment, the opening is large, e.g. forming more than 180 degrees of a circumference of a cross section of the distal end of the elongated member.

In this embodiment, the distal end of the elongated member may form a support extending less than 180 degrees of the circumference of a circle when seen in a cross section perpendicular to the longitudinal axis. This may allow the image capturing structure to move radially away from, and radially towards, the support. The support may e.g. be made by removing at least 180 degrees of the circumference of the aforementioned axial bore or by removing at least 180 degrees of the circumference of a distal portion of the axial bore.

The radial opening may be covered by a sheath which is elastically deformable and therefore allows movement of the image capturing structure during elastic deformation of the sheath.

In one embodiment, the sheath covers the entire circumference of the distal end of the elongated member or covers the entire elongated member.

The sheath, and/or other parts of the elongated member could be covered with a low friction material, e.g. a hydrophilic material, with Polytetrafluoroethylenes, PVC, or with any similar low friction material.

In one embodiment, the image capturing structure is not connected to the distal end of the elongated member and thereby becomes movable relative thereto, and in another embodiment, the image capturing structure and the distal end of the elongated member is connected by elastically deformable means. In one embodiment, the image capturing structure is connected to the elongated member by an elastically deformable polymer material e.g. silicone etc. The elastically deformable means may be constituted by the aforementioned sheath which, e.g. could be molded onto the distal end of the elongated member and onto the image capturing structure and thereby bond these entities elastically.

The device may comprise a valve structure configured to seal between an inner surface of the elongated conduit and an outer surface of an instrument arranged in the elongated conduit. The valve structure may include a duckbill valve or any similar kind of simple and reliable valve structure. The valve structure may e.g. be located in the control unit outside the elongated member.

The image capturing structure may be constituted by a lens located at the distal end, and the device may comprise fiber optical cables or other cables, extending from the lens through the elongated conduit to a camera inside the control unit. In one embodiment, the image capturing structure is constituted by a camera located at the distal end, e.g. a camera with an electronic circuit for converting the image into an electrical signal. In this embodiment, the captured images could be transmitted by electrical cables through the elongated conduit to further electronic processing in the control unit.

The term “Camera”, herein, covers any kind of structure for capturing an image or a series of images, e.g. for making a video sequence. Particularly, the camera may include a CCD, CMOS chip, lens, and other elements known in the art for capturing images.

Additionally, the image capturing structure, or the distal end of the elongated member may include illumination means, e.g. including one or more LEDs. In one embodiment, the device comprises illumination means capable of changing the color of the light. This may be useful for detecting various conditions of the uterus lining since certain frequencies provides better visibility for certain conditions.

The image capturing structure may particularly be configured to communicate the image in the form of electrical signals in analogue form. This alleviates the need for digital electronics at the distal end of the elongated member and thereby enables a more compact design which is desirable for providing an easier and potentially less painful access through the cervix.

The device may comprise heating means configured to heat the elongated member. Due to the increased surface temperature of the elongated member, the device can be used directly without further preparation and directly from the package without having to pre-treat or heat the device. The increased temperature makes insertion through cervix more comfortable.

A fluid flow provided in the vicinity of the image capturing structure may increase the quality of the images and may enable capturing of images through cervix and in the uterus. The device according to the invention may comprise handling means for handling release of a fluid medium at the distal end of the elongated member. These handling means may e.g. be located in the control unit.

In one embodiment, the handling means comprises a pump for pumping the fluid through the elongated member, e.g. through a hose extending through the elongated conduit. At the distal end, the device may comprise at least one release opening, e.g. in the form of one or more nozzles for release of the fluid. Particularly at least one of the nozzles may direct the fluid across the image capturing structure to prevent blood or tissue from blocking and preventing capturing of images.

The release opening may particularly be located in the forward or intermediate s-portions or between the rearward s-portion and the intermediate S-portion.

The pump may particularly be located in the control unit and it may be connectable to a source of fluid.

The heating means may be configured to heat the fluid medium e.g. to a temperature in the range of 35-38 degrees Celsius.

It may be an advantage to enable reorientation of the image capturing structure relative to the control unit to thereby enable change of the orientation of the image compared to the hand grip of the surgeon. For this purpose, the elongated member or at least a distal portion thereof may be rotatably relative to the hand piece. It may e.g. be connected through a swivel to the hand piece. The swivel may allow rotation of the elongated member and it may handle rotation of the hoses the aforementioned fluid and rotation of electrical or optical cables which extend from distal end of the elongated member to the control unit.

The elongated member may at least be able to rotate through a part of a circle, e.g. 10-90 degrees or even up to 180 degrees or more. To protect the hoses, cables, and other elements extending across the transition between the elongated member and the control unit, the rotation may be limited by a mechanical structure which stops further movement and therefore constitutes a stop member.

The electrical wires and hoses conducting signals and/or fluid through the elongated member may form slack portions enabling rotation of the elongated member relative to the control unit.

The device may comprise a release structure configured for releasable assembling of the control unit to the elongated member. The release structure may include valve means configured to prevent a fluid flow out of at least one of the control unit and the elongated member when the control unit and the elongated member are not assembled, and to allow the fluid flow once assembled.

The device may further comprise a biopsy implement configured to take biopsy samples from the uterus at the distal end of the elongated member.

The biopsy implement may comprise transfer means configured for transfer of taken biopsies from the distal end to the control unit.

The control unit may include an electrical component configured to control the camera and the monitor, and it may further comprise wireless communication means for communicating control commands and/or images with external devices.

The device may further comprise a structure allowing reorientation of the monitor relative to the elongated member.

The device may further comprise pressure means configured to distend the uterus by pressurization with a pressurized fluid. In one embodiment, the elongated member comprises a sealing member configured to seal against the cervix, and the aforementioned pump for administering a fluid flow is adapted to provide a pressure which is sufficient for distending the uterus and thereby provide a better visualization of the lining.

In a second aspect, the invention provides a device for visualization of internal tissue of a patient's uterus, the device comprising a hand-held control unit and an elongated member, the control unit dimensioned to be grasped by a user's hand and including a monitor, the elongated member having a proximal end in irremovably fixed connection with the hand-held control unit and a distal end with a camera where the camera is configured to communicate video signals with the monitor, and wherein the elongated member is dimensioned for insertion into the patient's uterus.

In a third aspect, the invention provides an internal tissue visualization device comprising a hand-held control unit and an elongated member, the control unit including a monitor and the elongated member having a proximal end fixed connected to the hand-held control unit and a distal end with a camera, where the camera is configured to communicate image data to the control unit, and where the control unit is configured to compensate for different orientations of the camera relative to a horizontal plane.

In one embodiment the elongated member is rotatable connected to control unit. This feature allows the user to rotate camera to a desired angle and still maintain a comfortable position of the handle, or a position of the handle that does not obstruct operation of an instrument through the tool conduit in the tube. Further the elongated member configured with a gripping member on a position on the elongated member adjacent the control unit.

In a fourth aspect, the invention provides a method of inspecting a uterus by use of a device as described herein.

### DETAILED DESCRIPTION

In the following, embodiments of the invention will be described in further details with reference to the drawing in which:

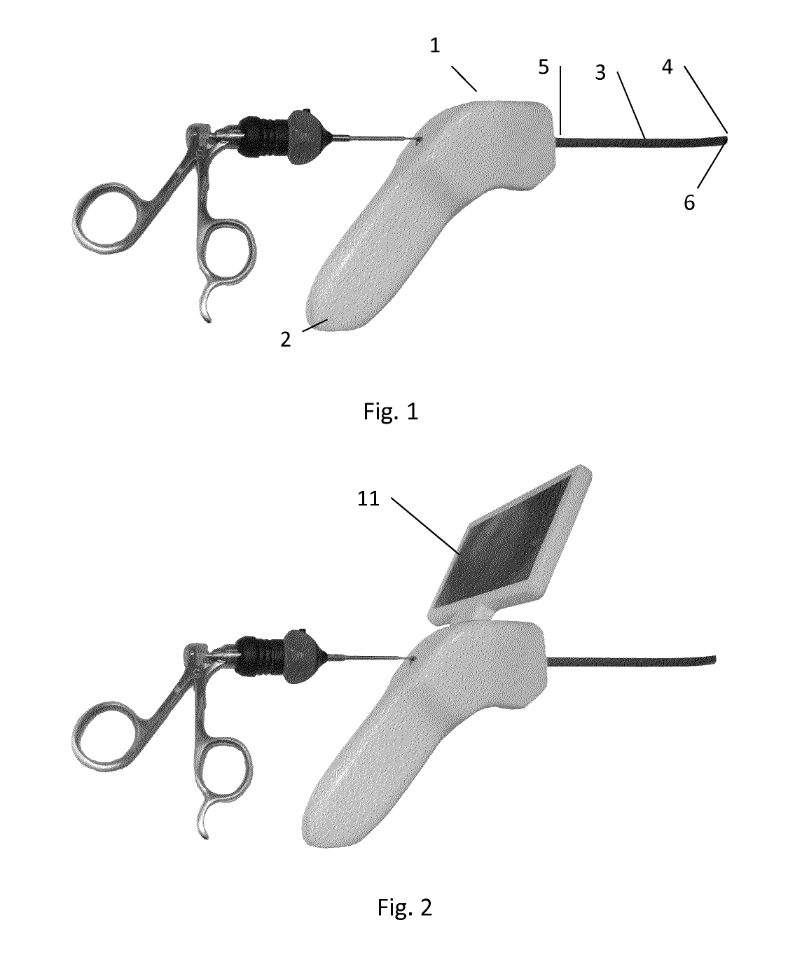

FIG. 1 illustrates an internal tissue visualization device according to the invention and a tool inserted in an elongated conduit;

FIG. 2 illustrates an internal tissue visualization device comprising a monitor mounted on the control unit according to the invention;

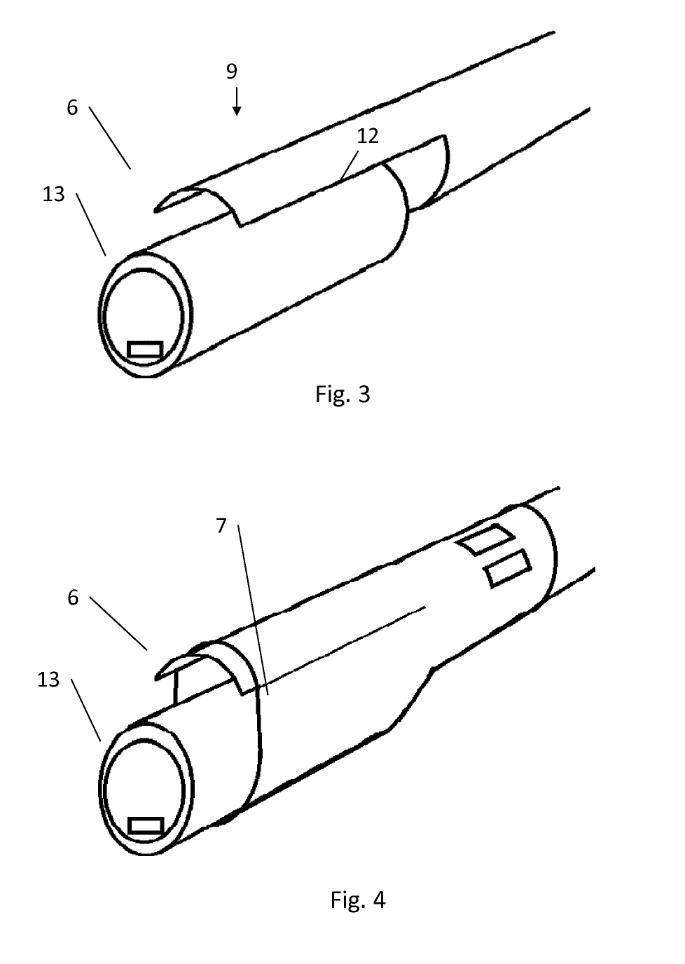

FIG. 3 illustrates the image capturing structure at the distal end of the elongated member;

FIG. 4 illustrates the distal end of the elongated member in an embodiment where the image capturing structure 6 is displaced from a center axis to provide space for an elongated conduit for the tool;

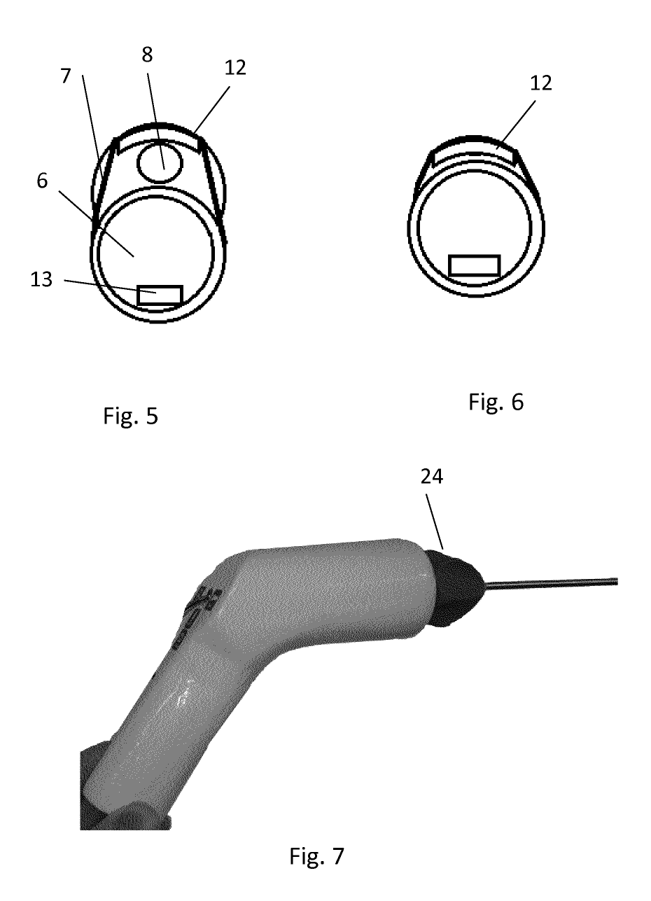

FIG. 5 illustrates the image capturing structure seen from the distal end image capturing structure and being displaced to provide unhindered passage for the tool through the elongated conduit;

FIG. 6 illustrates the image capturing structure from the distal end image capturing structure and not being displaced;

FIG. 7 illustrates an embodiment comprising the knob for rotating the elongated member 3 relative to the control unit;

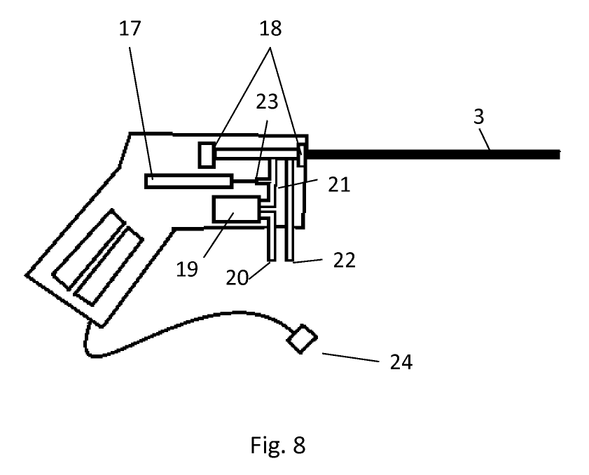

FIG. 8 illustrates internal components;

FIG. 9 illustrates an embodiment of the device with an S-shaped portion of the elongated member in a perspective view;

FIG. 10 illustrates the embodiment of FIG. 9 in a cross sectional view; and

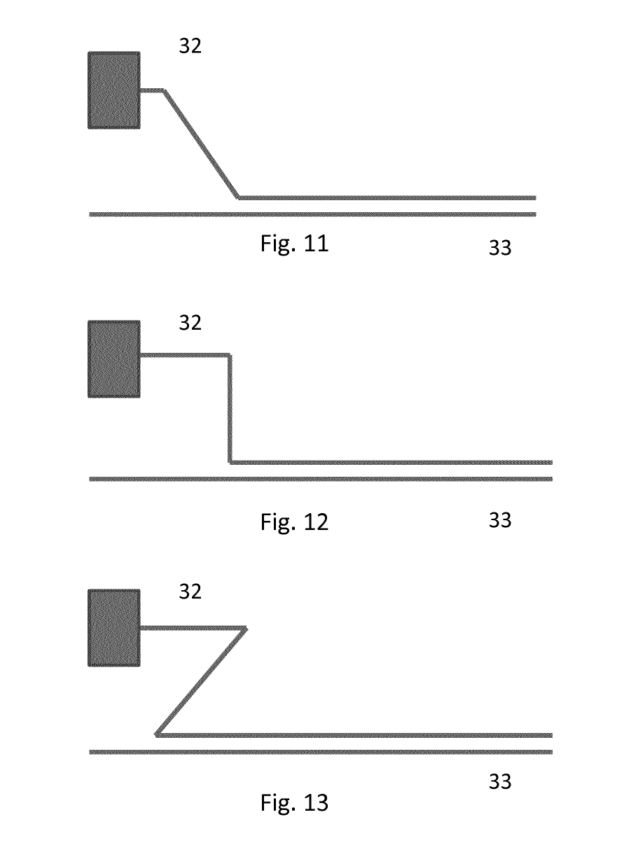

FIGS. 11-13 illustrate different shapes being considered within the definition of an S-shape.

It should be understood that the detailed description and specific examples, while indicating embodiments of the invention, are given by way of illustration only, since various changes and modifications within the spirit and scope of the invention will become apparent to those skilled in the art from this detailed description.

The tissue visualization device comprises an image capturing structure configured to capture picture of a desired tissue. The image capturing structure may be configured for non-electrical capturing or for electrical capturing of pictures. An example of an image capturing structure for non-electrical capturing is a lens by which the picture is transferred through the elongated member to a camera in the control unit. An example of an image capturing structure for electrical capturing is a camera, e.g. based on one or more CCDs sensitive in one or more wave length. The electrically captured picture is transferred by electrical cables through the elongated member to the control unit. The pictures could be still pictures and/or video by use of any method and format known per se.

The device comprises an elongated 3 member extending from a proximal end 5 to a distal end 4, the image capturing structure 6 is disposed at the distal end of the elongated member. The elongated member is fixed to the control unit, but it could also be releasable from the control unit.

The control unit 1 forms a housing for different electronic components. Particularly, the control unit houses a battery, a controller for processing data from the image capturing structure, and optionally communication means, e.g. comprising wireless transmission means, for transmitting the pictures to external systems. The control unit may further house a fluid pump 17 and pressure sensor. Further, the control unit may house heating means for heating fluid and/or for heating the elongated member. Further the control unit forms a handle 2 adapted to fit in the hand of the user, further the control unit 1 comprises an interface where the user can interact with the electronics, e.g. for controlling settings of the image capturing structure, pumping pressure, and heating etc.

The distal end holds the image capturing structure. In one embodiment, the image capturing structure is movable relative to the elongated member. The movability of the image capturing structure may be obtained e.g. by suspending the image capturing structure via an elastically deformable structure, e.g. a rubber belt or by a spring structure.

Generally, the elongated member may be rigid and dimensionally stable such that it forms a good support for tools in the elongated conduit and such that it is insertable e.g. through cervix and such that the image capturing structure can be manipulated by manipulation of the control unit. The elongated member could e.g. be made of a rigid material such as metal or a plastic.

FIG. 3 illustrates an embodiment where the distal end of the elongated member forms a cut-away section 9 starting from the distal end and extending inwardly. In the cut-away section, the distal end of the elongated member forms an extended member 12 which covers at most 180 degrees of the circumference thereby leaving at least 180 degrees free. In this embodiment, the image capturing structure 6 may be disposed in the cut-away section 9 adjacent, and supported by the extended member 12. An elastic deformable member 7 is disposed around the image capturing structure and around the extended member 12. The elastic deformable member 7 may form a ring-shape, it may e.g. be in the form of a tube or a sheath extending from a point near the distal end and covering at least the cut-away section. The elastic deformable member 7 is configured to hold the image capturing structure 6 in such a way that it is biased towards the extended member 12.

The elongated conduit may form a guide for tools to be inserted through the elongated member. The conduit extends between opposite proximal and distal openings. In the embodiment illustrated in FIG. 5, the distal opening 8 is illustrated. The image capturing structure 6 and the extending member 12 are configured such that a tool which is introduced in the elongated conduit will displace the image capturing structure relative to the extended member so that a passage is formed for the tool to pass the image capturing structure. An LED 13 is arranged to illuminate uterus.

The elongated member 3 is relatively long and slim to reduce discomfort for the patient during insertion. The elongated conduit may be used for inserting tools such as a scissor, a forceps or a morcellator etc.

Further the elongated member 3 comprises at least one conduit allowing fluid to be introduced e.g. into the utero during the surgery. The fluid is injected though an opening at the distal end. Such fluids may typically be injected to expand the uterus during a medical procedure or it may be injected to flush clear the image capturing structure and thereby create a clear sight. The elongated member 3 may include a second, fluid conduit that allows the fluid from the uterus to flow out through the elongated member 3, e.g. for collection at the control unit and/or for collection in an external reservoir. This will enable circulation of the fluid in the uterus and provide good visibility in the uterus.

According to the invention the control unit comprises an interface where the user can control different functions related to the image capturing structure, to the pump and fluid flow, to heating of the fluid and/or the elongated shaft, or for controlling bending of the elongated shaft. As an example, the control unit may house different electronics for image processing and for storage and transmission of data, and the user interface may allow picture saving, record video, zoom, light intensity and desired pump pressure. The interface may comprise push buttons, toggle keys, a touch screen, or similar interaction means.

Referring to FIG. 8, the control unit may contain different components. A pump 19 may be disposed in the control unit and configured for pumping fluid from an external reservoir into the uterus. The pump is provided with an inlet 20 and an outlet 21. The outlet is connected to a first fluid conduit through the elongated member 3, and the inlet forms an external connector outside the control unit making the pump connectable to an external reservoir containing the fluid. A second fluid conduit through the elongated member 3 ends in an external outlet 22 for release of fluid from the uterus.

A pressure sensor 23 may be provided between the pump and the first fluid conduit. Both the pump and the pressure sensor are connected to the electrical circuit 17 in the control unit. The electrical circuit 17 is configured to control the pump in relation to the pressure. For this purpose, it reads signals from the pressure sensor 21 such that a fixed or at least a predetermined pressure can be maintained in the utero. The pressure could be controlled by the user or it could be pre-programmed into the electronic circuit.

In one embodiment, the elongated member is rotatable relative to the control unit. The elongated member extends at least partially through the control unit, where the elongated member 3 is suspended in bearings 18 fixed within the control unit. The elongated member is provided with a toggle wheel 24 (Cf. FIG. 7) outside adjacent the control unit, that allows the user to easily manipulate the angular position of the elongated member 3. The toggle wheel may have a number of protrusions that allows a firm grip; The toggle wheel 24 is located close to the handle and can therefore be toggled with a finger of that hand which holds the control unit.

To allow the image capturing structure to communicate with the control unit and the pump to be in fluid communication with the conduits in the elongated member, the elongated member is connected by electrical wires and fluid conduits. Said connection is configured to allow the elongated member 3 to rotate freely without obstructing the electrical or fluid communication.

According to the invention the fluid connection might constitute a slack tube that allows the elongated member 3 to rotate at least a part of full rotation alternatively the elongated member 3 may be fitted with a fluid swivel connection. The electrical connection might constitute a slack wire that allows the elongated member 3 to rotate at least a part of a full rotation.

In one embodiment the tissue visualization device comprises a monitor 11 for displaying pictures captured by the image capturing structure and/or for user interaction, e.g. for adjusting different settings. The monitor could be a touch screen, and it could be releasably connected to the control unit.

The control unit may comprise a radio transmitter and the control unit may comprise an electrical circuit configured to transform the image signal from the image capturing structure into a radio signal for transmission to an external device. The external device could be a monitor. The connector 25 enables wired connection to a power supply and/or it enables transmission of data to external devices. The wired connection could be for the video signal to be transmitted, and it could therefore be a DVI cable, a HDMI, a Composite cable or other suitable standard cable.

FIGS. 9 and 10 illustrate an S-shaped portion 26 of the elongated member. Due to the S-shape, the image capturing structure 6 and the distal opening 8 of the elongated conduit becomes off-set by the distance indicated by the arrow 27. A tool which is in the elongated conduit may therefore pass the image capturing structure unhindered.

The S-shaped portion forms a forward S-portion the extend of which is illustrated by arrow 28, and a rearward S-portion the extend of which is illustrated by arrow 29. The forward and rearward S-portions are joined by an intermediate S-portion the extend of which is illustrated by arrow 30. The intermediate S-portion extends transverse to the forward and rearward S-portions.

In the illustrated embodiment, the forward and rearward S-portions extend essentially in parallel, and the rearward S-portion is in line with the remaining part of the elongated member. Accordingly, the rearward S-portion may be completely integrated in the remaining elongated member such that no visual transition exists.

The elongated member of this embodiment of the device comprises a proximal portion forming the proximal end and a distal portion forming the distal end, the proximal portion and the distal portion having different bending characteristics. More particularly, the rearward S-portion has a reduced bending moment as compared to the bending moment of the remaining parts of the elongated member. The reduced bending moment is established by the illustrated slots 31. Alternatively, the reduced bending moment could be established by different material characteristics, particularly relative to elastic deformation and/or by different dimensions.

In the illustrated embodiment, the forward S-portion and the rearward S-portion obtains different bending characteristics due to the aforementioned slots 31.

the distal opening 8 is between the rearward S-portion 29 and the intermediate S-portion 30.

FIGS. 11-13 illustrate three different lines 32 illustrating the shape of the elongated member 3. In FIG. 11, the intermediate S-portion 30 points in a forward direction towards the distal end of the elongated member, in FIG. 13, the intermediate S-portion 30 points in a rearward direction towards the proximal end of the elongated member, and in FIG. 12 the intermediate S-portion is perpendicular to the forward and rearward S-portions. The Angle of the intermediate S-portion relative to the forward and rearward S-portions may particularly be less than plus or minus 70 degrees from perpendicular, such that the intermediate S-portion forms an angle between 20 and 170 degrees to the longitudinal direction of the elongated member. The longitudinal direction is illustrated by the line 33. The line 33 also illustrates the direction of a tool through the internal conduit.

### LIST OF NUMBERED EMBODIMENTS

1. A device for visualization of internal tissue of a patient's uterus, the device comprising a hand-held control unit, an elongated member, and an image capturing structure, the control unit being dimensioned to be held by a user's hand and including a monitor, the elongated member having a proximal end connected to or connectable to the hand-held control unit and a distal end holding the image capturing structure, where the image capturing structure is configured to communicate video signals with the monitor, and where at least the distal end of the elongated member and the image capturing structure is dimensioned for insertion into the patient's uterus through cervix.

2. A device according to embodiment 1, where the image capturing structure is movable relative to the distal end of the elongated member.

3. A device according to embodiment 2, where the image capturing structure is connected to the distal end by elastically deformable means.

4. A device according to any of the preceding embodiments, where the elongated member forms an elongated conduit between a proximal opening and a distal opening.

5. A device according to embodiment 4, comprising a valve structure configured to seal between an inner surface of the elongated conduit and an outer surface of an instrument arranged in the elongated conduit.

6. A device according to any of embodiments 2-3 and any of embodiments 4-5, where the image capturing structure is movable between a first position in which it at least partly hinders passage through the elongated conduit and a second position providing unhindered passage through the elongated conduit.

7. A device according to any of the preceding embodiments, where the image capturing structure comprises a camera and an electronic circuit for converting the image into an electrical signal.

8. A device according to embodiment 7, where the electrical signal is a communicated in analogue form.

9. A device according to any of the preceding embodiments, comprising heating means configured to heat the elongated member.

10. A device according to any of the preceding embodiments, where the control unit comprises handling means for handling release of a fluid medium at the distal end of the elongated member.

11. A device according to embodiment 10, where the handling means comprises a pump.

12. A device according to embodiment 10 or 11, comprising heating means configured to heat the fluid medium.

13. A device according to any of embodiment 1-12 where the elongated member is rotatably connected to the hand piece.

14. A device according to embodiment 13 where the rotatable elongated member can be rotated at least a part of a circle, and where the rotation is limited by stop members.

15. A device according to embodiment 13 or 14 where rotatable elongated member is connected to the control unit with at least one electrical wire to allow the control unit to communication with the image capturing structure,

16. A device according to any of embodiments 13-15, where rotatable elongated member is connected to the control unit with at least one hose to allow the control unit to communicate fluid communication with the elongated member.

17. A Device according to any of embodiments 15 or 16, where at least one of the wires or one of the hoses are slack to allow rotation of the elongated member relative to the control unit.

18. A device according to any of embodiments 15-17, comprising a swivel forming a fluid outlet out of the control unit and a fluid inlet into the elongated member, the swivel being configured to that maintain a stationary position of the fluid outlet relative to the control unit when the elongated member is rotated relative to the control unit.

19. A method of carrying out hysteroscopy by use of a device according to any of the preceding embodiments, the method comprising the step of heating the elongated member, the subsequent step of inserting the elongated member into uterus of the patient, and the step of capturing images in the uterus by use of the camera.

20. A device for visualization of internal tissue of a patient's uterus, the device comprising a hand-held control unit and an elongated member, the control unit dimensioned to be grasped by a user's hand and including a monitor, the elongated member having a proximal end in irremovably fixed connection with the hand-held control unit and a distal end with a camera where the camera is configured to communicate video signals with the monitor, and wherein the elongated member is dimensioned for insertion into the patient's uterus.

21. An internal tissue visualization device comprising a hand-held control unit and an elongated member, the control unit including a monitor and the elongated member having a proximal end fixed connected to the hand-held control unit and a distal end with a camera, where the camera is configured to communicate image data to the control unit, and where the control unit is configured to compensate for different orientations of the camera relative to a horizontal plane.

1-23. (canceled)

24. A device for visualization of internal tissue of a patient's uterus, the device comprising a hand-held control unit, an elongated member, and an image capturing structure, the control unit being dimensioned to be held by a user's hand, the elongated member having a proximal end connected to or connectable to the hand-held control unit and a distal end holding the image capturing structure, where the image capturing structure is configured to communicate video signals representing still pictures or video with a monitor, where at least the distal end of the elongated member and the image capturing structure is dimensioned for insertion into the patient's uterus through cervix, where the control unit comprises handling means for handling release of a fluid medium at the distal end of the elongated member, and where the handling means comprises a pump located in the control unit.

25. The device according to claim 24, wherein the pump is arranged for pumping the fluid through the elongated member.

26. The device according to claim 24, comprising at least one release opening at the distal end of the elongated conduit for release of the fluid medium.

27. The device according to claim 24, wherein the pump is connectable to an external source of fluid.

28. The device according to claim 24, where the elongated member forms an S-shaped portion.

29. The device according to claim 28, wherein the elongated member forms an elongated conduit between a proximal opening and a distal opening, and where the distal opening is in the S-shaped portion.

30. The device according to claim 28, comprising at least one release opening for release of the fluid medium, the release opening being located in a forward or an intermediate s-portions or between a rearward s-portion and the intermediate S-portion.

31. The device according to claim 30, wherein at least one release opening forms a nozzle directing the fluid medium across the image capturing structure to prevent blood or tissue from blocking and preventing capturing of images.

32. The device according to claim 24, wherein the elongated member comprises a sealing member configured to seal against the cervix, and the pump is configured to provide a pressure which is sufficient for distending the uterus and thereby provide a better visualization of the lining.

33. The device according to claim 24, wherein the control unit houses heating means for heating the fluid medium.

34. The device according to claim 24, comprising a second fluid conduit configured to allow the fluid medium to flow from the uterus out through the elongated member.

35. The device according to claim 24, further comprising a pressure sensor and an electronic circuit configured to control the pump in relation to the pressure.

36. The device according to claim 24, where the elongated member is rotatably connected to the control unit.

37. The device according to claim 36, where the rotation is limited by mechanical stop members.

38. The device according to claim 36, where the rotatable elongated member is connected to the control unit with at least one electrical wire to allow the control unit to communication with the image capturing structure, where the rotatable elongated member is connected to the control unit with at least one hose to allow the control unit to communicate fluid communication with the elongated member, and where at least one of the wires or one of the hoses are slack to allow rotation of the elongated member relative to the control unit.

39. The device according to claim 24, where the control unit comprises a monitor.
